SEQUENCE LISTING (58 sequences)

SEQ ID NO: 1 - protein, 15 aa, Artificial sequence
  peptide
MATIRTVRNLKLCNP

SEQ ID NO: 2 - protein, 15 aa, Artificial sequence
  peptide
TRLLTVVVKQRSKAF

SEQ ID NO: 3 - protein, 15 aa, Artificial sequence
  peptide
LTVVVKQRSKAFRPS

SEQ ID NO: 4 - protein, 15 aa, Artificial sequence
  peptide
RVSYGSECRSGNCLR

SEQ ID NO: 5 - protein, 9 aa, Artificial sequence
  peptide
ATIRTVRNL

SEQ ID NO: 6 - protein, 15 aa, Artificial sequence
  peptide
LDDFLSLQRSISPNS

SEQ ID NO: 7 - protein, 15 aa, Artificial sequence
  peptide
ATHYFEMTSDAFFAD

SEQ ID NO: 8 - protein, 15 aa, Artificial sequence
  peptide
DDFLSLQRSISPNSY

SEQ ID NO: 9 - protein, 15 aa, Artificial sequence
  peptide
DVKIAVDPACKLSAR

SEQ ID NO: 10 - protein, 15 aa, Artificial sequence
  peptide
HLILRITPGIDLREL

SEQ ID NO: 11 - protein, 15 aa, Artificial sequence
  peptide
LNQLVSLVKQIFVQQ

SEQ ID NO: 12 - protein, 15 aa, Artificial sequence
  peptide
QLMYLIEITSETKAL

SEQ ID NO: 13 - protein, 15 aa, Artificial sequence
  peptide
GRYYAEINSAKMYFG

SEQ ID NO: 14 - protein, 15 aa, Artificial sequence
  peptide
VLVLVGDEAAALEQL

SEQ ID NO: 15 - protein, 15 aa, Artificial sequence
  peptide
AAALEQLLGQTADVA

SEQ ID NO: 16 - protein, 9 aa, Artificial sequence
  peptide
QLLGQTADV

SEQ ID NO: 17 - protein, 15 aa, Artificial sequence
  peptide
RRTVKILRPLPSNIT

SEQ ID NO: 18 - protein, 15 aa, Artificial sequence
  peptide
NHAVSSDFKTRSTNT

SEQ ID NO: 19 - protein, 9 aa, Artificial sequence
  peptide
DIAARLASL

SEQ ID NO: 20 - protein, 15 aa, Artificial sequence
  peptide
FDEYRAMFALSAMDL

SEQ ID NO: 21 - protein, 15 aa, Artificial sequence
  peptide
THAFDLVLSSHLLFT

SEQ ID NO: 22 - protein, 15 aa, Artificial sequence
  peptide
DDLLARLRADGVHGE

SEQ ID NO: 23 - protein, 15 aa, Artificial sequence
  peptide
MLILGLLPAILPACG

SEQ ID NO: 24 - protein, 15 aa, Artificial sequence
  peptide
AILYRKIHGQDPAER

SEQ ID NO: 25 - protein, 9 aa, Artificial sequence
  peptide
LILGLLPAI

SEQ ID NO: 26 - protein, 15 aa, Artificial sequence
  peptide
PYRFHSLAAIWALSP

SEQ ID NO: 27 - protein, 15 aa, Artificial sequence
  peptide
GINLPKDELTAFGRK

SEQ ID NO: 28 - protein, 15 aa, Artificial sequence
  peptide
VGAVRIVGGVRPQNF

SEQ ID NO: 29 - protein, 15 aa, Artificial sequence
  peptide
EAVLLRLDGTTLEVE

SEQ ID NO: 30 - protein, 15 aa, Artificial sequence
  peptide
QVIFRDLTTQKAAEE

SEQ ID NO: 31 - protein, 15 aa, Artificial sequence
  peptide
SGRVTYLNPVGVKWM

SEQ ID NO: 32 - protein, 9 aa, Artificial sequence
  peptide
LLEEGVIVL

SEQ ID NO: 33 - protein, 9 aa, Artificial sequence
  peptide
RLDGTTLEV

SEQ ID NO: 34 - protein, 15 aa, Artificial sequence
  peptide
MQEYRGLTSHTPCCR

SEQ ID NO: 35 - protein, 15 aa, Artificial sequence
  peptide
LDIYTTLARDMAAIP

SEQ ID NO: 36 - protein, 15 aa, Artificial sequence
  peptide
TIEFLWQEGHSAHIE

SEQ ID NO: 37 - protein, 15 aa, Artificial sequence
  peptide
DAEWLKLTSLGLRPR

SEQ ID NO: 38 - protein, 16 aa, Artificial sequence
  peptide
VKAVVDDVNAILLTGR

SEQ ID NO: 39 - protein, 15 aa, Artificial sequence
  peptide
LQPYRLLRGGDSEYW

SEQ ID NO: 40 - protein, 15 aa, Artificial sequence
  peptide
VGKVQGLLARLLTLP

SEQ ID NO: 41 - protein, 15 aa, Artificial sequence
  peptide
DSTDTTDWSTAMTDA

SEQ ID NO: 42 - protein, 15 aa, Artificial sequence
  peptide
GLDSIISSASASLLT

SEQ ID NO: 43 - protein, 15 aa, Artificial sequence
  peptide
GNAMTTLGGRGISVV

SEQ ID NO: 44 - protein, 15 aa, Artificial sequence
  peptide
YGQVVRDVENTLRYL

SEQ ID NO: 45 - protein, 15 aa, Artificial sequence
  peptide
GDVWKSIVHLRSTRH

SEQ ID NO: 46 - protein, 15 aa, Artificial sequence
  peptide
KKRLTLLSGAMARRA

SEQ ID NO: 47 - protein, 15 aa, Artificial sequence
  peptide
DATFQVIFSQHVHLS

SEQ ID NO: 48 - protein, 15 aa, Artificial sequence
  peptide
MIDYNNVFGAGVVAA

SEQ ID NO: 49 - protein, 15 aa, Artificial sequence
  peptide
NYEVSPIFARWPRNR

SEQ ID NO: 50 - protein, 15 aa, Artificial sequence
  peptide
VYLYNYLLAETSHVL

SEQ ID NO: 51 - protein, 15 aa, Artificial sequence
  peptide
YRYYRLIATTDAASP

SEQ ID NO: 52 - protein, 15 aa, Artificial sequence
  peptide
DDIWRTLASAVITGN

SEQ ID NO: 53 - protein, 15 aa, Artificial sequence
  peptide
FDSFDRILKARSPEA

SEQ ID NO: 54 - protein, 15 aa, Artificial sequence
  peptide
KENVIIVAAKISWTL

SEQ ID NO: 55 - protein, 15 aa, Artificial sequence
  peptide
LATVQYDDRRRFTKE

SEQ ID NO: 56 - protein, 15 aa, Artificial sequence
  peptide
LQAYSNLFGRTSAMQ

SEQ ID NO: 57 - protein, 15 aa, Artificial sequence
  peptide
GMDVGKVEALKIDGD

SEQ ID NO: 58 - protein, 15 aa, Artificial sequence
  peptide
WASVALLTAAGVSLG

(19) United States
(12) Patent Application Publication
(10) Pub. No.: US 2008/0146511 A1
(43) Pub. Date: Jun. 19, 2008

(54) SELECTION METHOD OF PEPTIDES, PEPTIDES, METHOD AND KIT OF INFECTION IDENTIFICATION BY MYCOBACTERIUM LEPRAE, PHARMACEUTICAL OR IMMUNOLOGICAL COMPOSITIONS CONTAINING PEPTIDES OF MYCOBACTERIUM LEPRAE OR SEQUENCES FUNCTIONALLY EQUIVALENT

(75) Inventors: Maria Cristina Vidal Pessolani, Rio De Janeiro (BR); Geraldo Moura Batista Pereira, Rio De Janeiro (BR); Marcia Valeria Brandao dos Santos Martins, Rio De Janeiro (BR); Monica Cristina Brandao dos Santos Lima, Rio De Janeiro (BR); Patrick Joseph Brennan, Fort Collins, CO (US); John Stewart Spencer, Fort Collins, CO (US); Hazel Marguerite Dockrell, London (GB)

Correspondence Address: KLARQUIST SPARKMAN, LLP, 121 SW SALMON STREET, SUITE 1600, PORTLAND, OR, 97204, US

(73) Assignee: Fundacao Oswaldo Cruz - FIOCRUZ

(21) Appl. No.: 11/611,119

(22) Filed: Dec. 14, 2006

(30) Foreign Application Priority Data
Dec. 14, 2006 (BR) 20060185350

(51) Int. Cl. A61K 38/00; A61P 43/00; C07K 7/00; G01N 33/48; G01N 33/554

(52) U.S. Cl. 514/14; 435/7.32; 436/86; 514/15; 530/326; 530/328

(57) ABSTRACT

The present invention refers to a selection method of peptides, peptides, method and kit for the identification of infections by Mycobacterium leprae, as well as to pharmaceutical and immunological compositions containing as active principle(s), peptide(s) of Mycobacterium leprae or sequences functionally similar to the same one(s).

### FIELD

The present invention refers to a selection method of peptides, peptides, method and kit for the identification of infections by Mycobacterium leprae, as well as to pharmaceutical and immunological compositions containing as active principle (s), peptide(s) of Mycobacterium leprae or sequences functionally similar to the same one(s).

### BACKGROUND

The first descriptions of Hansen's disease (or “leprosy”, as it was known historically), and the first reports of people with characteristic symptomatology of the disease date from 600 B.C.—in India (Maurano, F. Tratado de leprologia [Treaty of Leprology]. Vol. 1. História da Lepra no Brasil e sua distribuicão geográfica [History of Leprosy in Brazil and its geographical distribution]. Ministério da Educacão e Saúde [Ministry of Education and Health]. Departamento Nacional de Saúde [National Department of Health]. Servico Nacional de Lepra. [National Leprosy Service]. Rio de Janeiro, 1944. CEDOPE/HCI. However, in the beginning of XIII century, leprosy was already very frequent in Europe, with the existence of approximately 19,000 leper colonies. And concerning the Americas, Hansen's disease must have arrived to these territories with European settlers, especially in Brazil, between XVI and XVII centuries (Opromolla, D. V. A. Nocões de Hansenologia [Notions of Hansenology]. Centro de Estudos [Center of Studies] Dr. Reynaldo Quagliato. Ed. 1. São Paulo: 2000. pages 101-115).

As of XVII century, Hansel's disease has diminished in Europe, probably due to the isolation of patients, sanitation improvements and raising of the population standard of living (Maurano, F. Tratado de leprologia. Vol. 1. História da Lepra no Brasil e sua distribuicão geográfica. Ministério da Educacão e Saúde. Departamento Nacional de Saúde. Servico Nacional de Lepra. Rio de Janeiro, 1944. CEDOPE/HCI). On the other hand, currently all South American countries—except for Chile—have Hansen's disease (Opromolla, D. V. A. Nocões de Hansenologia. Ed. 1. São Paulo: Centro de Estudos Dr. Reynaldo Quagliato, 2000. Pages 101-115). And, at worldwide level and according to data of the World Health Organization, Hansen's disease is more concentrated in nine countries, in which it still represents a problem of public health. Together, these countries (Angola, Brazil, Central African Republic, Democratic Republic of Congo, India, Madagascar, Mozambique, Nepal and United Republic of Tanzania) respond for 84% of the prevalence and 88% of the worldwide detection (World Health Organization, Leprosy Today, 2004, available on the internet at http://who.int/lep/).

The disease in question is infectious, of chronic evolution, caused by the bacterium Mycobacterium leprae (known as ‘Hansen's Bacillus’), which presents in human tissues as a straight rod cell or slightly bent, measuring approximately 1 up to 8 μm length by 0.2 up to 0.5 μm diameter (Rees R. J. W., Young D. B. In: Hastings R. C.; Opromolla D. V. A. Leprosy. Ed. 2, New Orleans: Churchill Livingstone, 1994, p. 49-76). The location of Hansen lesions in the patients' body (skin, mucous membranes and peripheral nerves) suggests that the bacillus has preference for temperatures lower than 37° C. (Lombardi C. Hanseníase: epidemiologia e controle [Hansen's Disease: epidemiology and control]. São Paulo. IMESP. 1990. Page 23).

The main symptoms of the disease are white or reddish stains, pain in the nerves of the arms, hands, legs or feet and body parts with tingling or numbness. Usually, the victim of Hansen's disease also presents cutaneous eruptions in the body, absence of pain in cases of burns or cuts in the arms, hands, legs and feet (Ministério da Saúde [Ministry of Health], Tópicos de Saúde [Health Topics], Hanseníase [Hansen's Disease], 2006. Available on the internet at http://portal.saude.gov.br/portal/svs/visualizar_texto.cfm?idtxt=21643).

The clinical manifestations vary from the polar tuberculoid form (TT—individuals with hyperresponsivity to Mycobacterium leprae) up to the polar lepromatous form (LL—individuals hyporesponsive to the bacillus). There are intermediate forms: borderline tuberculoid (BT), borderline borderline (BB) and borderline lepromatous (BL). These forms can be roughly assembled in multibacillaries—MB (LL, BL and BB), and paucibacillaries—PB (TT and BT). Patients with multibacillary forms are considered the main source of infection (Riddley D. S., Joppling W. H. Classification of leprosy according to immunity a five-group system. Int. J. Lepr. 1966; 34: 255-73).

The infection caused by this pathogen presents a long period of latency, being four years the average time of incubation, approximately, in patients of the tuberculoid pole, and of up to 30 years for the ones of the lepromatous pole. This long period of incubation contributes to the transmission of Hansen's disease, because even before the clinical manifestation of symptoms, the individual is already a potential transmitter (Noordeen S. K. The epidemiology of leprosy. In: Hastings R C, ed. Leprosy, 2^{nd} Edn. Edinburg: Churchill-Livingstone, 1994:29-48; Pessolani M. C. V., Marques M. A. M., Reddy V. M., Locht C., Menozzi F. D. Systemic dissemination, a common trait in tuberculosis and leprosy. Microbes Infect. 2003; 5 (7): 677-84).

The contact with Mycobacterium leprae is mainly made by the upper airways, and subclinical infection occurs in a large ratio of exposed people (revised by Gallo M. E. N., Sampaio E., Nery J. A., Morais M. O., Antunes S. L., Pessolani M. C. V., Sarno E. N., EDITOR: Coura, J R. Hanseníase {Leprosy]: Aspectos Epidemiológicos, Clínicos e Imunológicos [Epidemiological, Clinical and Immunological Aspects]. Dinâmica das doencas infecciosas e parasitárias [Dynamics of infectious and parasitic diseases]. 2005. Editora [Publishing House] Guanabara Koogan S A. Pages:1383-1394). Almeida and collaborators have clearly demonstrated that DNA of this pathogen was present in the patients' mucous membrane and of their contacts, corroborating the idea that latent infection occurs frequently, and that the nose is the main exit and entrance pathway of Mycobacterium leprae (Almeida E. C., Martinez A. N., Manierom V. C., Sales A. M., Dupre N. C., Sarno E. N., Santos A. R., Moraes M. O. Detection of mycobacterium leprae DNA by polymerase chain reaction in the blood and nasal secretion of Brazilian household contacts. Mem. Inst. Oswaldo Cruz. 2004. 99: 509-511).

Concerning the vaccination against leprosy, one can mention that its prevention is made by means of the use of the Bacillus of Calmette-Guérin vaccine (BCG), the same one used for the prevention of tuberculosis. The BCG vaccine confers between 20 and 90% of protection against MB and PB forms of Hansen's disease in all populations, and one of the hypotheses for that variation is a possible crossed reaction with mycobacteria in the environment (Fine P E. The Kellersberger memorial lecture. The role of BCG in the control of leprosy. Ethiop Med J. 1985; 23:179-191; Fine P. E. M., Smith P. G. Vaccination against leprosy—The View from 1996; 67: 249-52; Lombard C. Hanseníase: epidemiologia e controle. São Paulo. IMESP. 1990, p. 23).

In Brazil, since 1992 the vaccination for leprosy with BCG was recommended by the Ministry of Health (Matos H., Duppre N., Alvin M. F. S., Vieira L. M. M., Sarno E. N., Struchiner C. J. Epidemiologia da hanseniase em coorte de contatos intradomiciliares no Rio de Janeiro [Epidemiology of Hansen's Disease in cohort of intradomicile contacts in Rio de Janeiro] (1987-1991). Cad. Saúde Pública. 1999; 15: 533-542). And currently the application of two doses of BCG is extolled in all healthy individuals, classified as intradomicile contacts of all new cases of leprosy, independently of the clinical form of case-index (Lombard C. Hanseníase: epidemiologia e controle. São Paulo. IMESP. 1990, p. 23. Pompeu A. F., Moraes A. C. R. Avaliacão da presenca de cicatriz vacinal da BCG e correlacão com as formas clínicas da hanseníase [Evaluation of the presence of vaccination scar of BCG and correlation with clinical forms of leprosy]. 2002. 30 f. Monografia (Graduacão em Medicina) [Monography (Graduation in Medicine)]—Centro de Ciências da Saúde [Center of Health Sciences], Universidade Estadual [State University] do Pará. Belém, 2002). It is also relevant to mention that in the Brazilian territory the strain BCG Moreau is used for manufacturing the vaccine in question. And, this strain has been conferring about 70% of protection against Hansen's disease in neonatal vaccination, mainly for the form MB (Cunha S. S., Rodrigues L. C., Pedrosa A., Dourado I. M., Barreto M. L., Pereira S. M. Neonatal BCG protection against leprosy: a study in Manaus, Brazilian Amazon. Lepr. Rev. 2004; 75, 357-366). In groups of risk (contacts), the vaccination with BCG has been conferring about 50% of protection, being this protection increased in 25% with the vaccine repetition (Smith C. M., Smith W. C. Chemoprofilaxis is effective in the prevention of leprosy in endemic countries: a systematic review and meta-analysis. J Infect. 2000; 41:137-42).

In relation to the use of chemotherapy agents in the treatment of leprosy, it refers to times gone by VI century. And, since the 40's the monotherapy with sulfones was adopted, thus causing a great impact in the disease treatment. Nevertheless the appearance of sulfone-resistant cases, the bacillary persistence and the treatment discontinuation due to its long duration have lead the WHO to recommend, in 1982, a polychemotherapy treatment based on the association of the drugs dapsone, rifampicin and clofazimine (revised by Gallo M. E. N., Sampaio E., Nery J. A., Morais M. O., Antunes S. L., Pessolani M. C. V., Sarno E. N., EDITOR: Coura, J R. Hanseníase: Aspectos Epidemiológicos, Clínicos e Imunológicos. Dinâmica das doencas infecciosas e parasitárias. 2005. Editora Guanabara Koogan S A. Pages:1383-1394).

Additionally, it is essential to highlight that despite the introduction of polychemotherapy (PQT) and vaccination with BCG, the number of new cases detected annually remains constant or has increased. This persistence in the detection rate of new cases of leprosy in endemic countries can be attributed to several factors, such as: to the intensity in the search of new cases, to the high transmission in certain areas caused by asymptomatic transmitters, or to the recurrence of cases previously treated and to the lack of confirmation of the disease in an early stage. Brazil presents approximately 45,000 new cases per year (www.saude.gov.br).

Therefore, in Brazil a worrying data is the maintenance of a high rate of detection of new cases of leprosy, notwithstanding the fall in the prevalence indexes, what suggests the permanence of a high rate of transmission in the population. Among new cases identified in this country, more than a half manifests one of the multibacillary forms of the disease, and a high percentage (approximately 8%) corresponds to children (younger than 14 years) and a significant number of cases detected already displays some degree of physical disability (World Health Organization, Leprosy Today, 2004, Available on the internet at http://who.int/lep/). This epidemic data indicates that the detection of leprosy is performed too late in Brazil, thus favoring the transmission chain.

So, in terms of public health, the early treatment would prevent the development of severe forms of the disease in question. Also the early confirmation followed by an adequate treatment could eliminate potential sources of transmission, thus contributing to the disruption of the epidemic chain and elimination of this disease.

In this sense, it is of utmost importance to emphasize that the high prevalence of tuberculosis in Brazil, for instance , allied to the obligatoriness of vaccination with BCG (which was obtained by attenuation of Mycobacterium bovis) in neonates require this test to be specific, detecting only individuals infected by the bacillus of Hansen, or better, Mycobacterium leprae.

So, the search for molecules of Mycobacterium leprae is being performed. By means of conventional biochemical methodologies it was possible to isolate and characterize a series of molecules of Mycobacterium leprae. Also, with the advent of molecular biology, the construction of genomic libraries of the bacillus, which were then traced with monoclonal and polyclonal antibodies, leading to the identification of a series of antigens. However several of these antigens, as the proteins of thermal shock 70, 65, 18, and 10 kDa, are present in other mycobacteria, as Mycobacterium tuberculosis and present large homology among them, not being so adequate for purposes of the presence confirmation of Mycobacterium leprae (Marques, M A M et al. Continued proteomic analysis of Mycobacterium leprae subcellular fractions. Proteomics 4: 2942-2953, 2004).

### SUMMARY OF THE DISCLOSURE

The main objective of the present invention is the specific identification of infections by M. Leprae, including those concerning latent forms or initial phases of active disease.

A first embodiment of the present invention refers to a selection method of peptides from proteins or regions of proteins belonging to the genome of M. leprae with algorithms for the detection of regions that combine with human molecules of histocompatibility and which are capable to induce an immune response in individuals exposed to M. leprae.

A second embodiment of the present invention is related to peptides p37 up to p94 (corresponding to SEQ ID No. 1 up to SEQ ID No. 58 respectively, or to sequences functionally equivalent to the same ones).

A third embodiment of the present invention refers to a method for sensitive and specific detection of infections by M. leprae through the following stages:

(a) Collect the sample to be examined;

(b) Extract from the sample obtained in (a) the biological material of interest;

(c) Put the biological material originating from stage (b) in contact with at least one of the peptides p37 up to p94 (corresponding to SEQ ID No. 1 up to SEQ ID No. 58 respectively, or to sequences functionally equivalent to the same ones);

(d) In view of the contact carried out in stage (c), by means of appropriate techniques detect antigen-specific induction of immunoinflammatory molecules and/or presence of cells responding to peptides used (or sequences functionally equivalent to the same ones).

A fourth embodiment of the present invention is related to a kit for use in sensitive and specific detection of infections by M. leprae through the method presented in the present invention. Basically, the kit comprises at least one of the peptides p37 up to p94 (corresponding to SEQ ID No. 1 up to SEQ ID No. 58 respectively, or to sequences functionally equivalent to the same ones), additives and reagents necessary for the execution of stage (c) of the present method, as in the case of buffers, monoclonal antibodies, culture medium, enzymes and enzymatic substrata, etc., in sufficient amounts to carry out said stage (c). Optionally, it may contain protocol and manual to give instructions to the user.

A fifth embodiment of the present invention is related to pharmaceutical or immunological compositions, which have as active principle (s) at least one of the peptides p37 up to p94 (corresponding to SEQ ID No. 1 to SEQ ID No. 58 respectively, or to sequences functionally equivalent to the same ones).

### BRIEF DESCRIPTION OF THE DRAWINGS

FIG. 1: Levels of IFN-γ in supernatants of PBMC cultures originated from PB, MB, HC, TB, EC and NEC patients in response to four recombinant proteins, to total cells of M. leprae and to SEB superantigens.

FIG. 2: Levels of IFN-γ in supernatants of PBMC cultures originated from PB, MB, HC, TB, EC and NEC patients in response to a representative subset of 58 peptides tested.

FIG. 3: Levels of IFN-γ in PB, TB and EC groups in response to four recombinant proteins, to their respective peptides, to the peptides from ML0394 protein and to total cells of M. leprae

FIG. 4: Frequencies of T CD4+ and CD8+ activated lymphocytes (CD69+) and producers of IFN-γ specific to nonamer (p52, p61, e p69) and decapentamer peptides (p51, p59, e p65) and their mixture.

FIG. 5: ANOVA of the levels of IFN-γ induced by synthetic peptides of Mycobacterium leprae.

### DETAILED DESCRIPTION OF THE INVENTION

The present invention aims at the specific identification of infections by M. leprae, including those concerning latent forms or initial phases of active disease.

The sequencing of the genome of M. leprae and of other pathogenic mycobacterium allowed the selection, in silico, of private proteins of M. leprae.

So, one of the concretions of the present invention refers to a selection method of peptides from proteins or regions of proteins belonging to the genome of M. leprae with algorithms for the detection of regions that combine with human molecules of histocompatibility, and which are capable to induce an immune response in individuals exposed to M. leprae. In the present invention, this immune response can be exemplified, but it is not limited to the immune response in vitro in leucocytes obtained from individuals exposed to M. leprae.

The analysis of these M. leprae-specific structures by means of appropriate algorithms using the SYFPEITHI program (www.bmi-heidelberg.com/syfpeithi) identified a great number of regions with potential association to HLA molecules of class I and class II. So, in the present invention, from this analysis, recombinant proteins were obtained, in addition to M. leprae-specific synthetic peptides with affinity to HLA of class I (nonamers) and of class II (pentadecamers).

Peptides from the same ones (p37 up to p94) are presented in Table 1 contained in the Examples. The respective sequences are provided in the Listing of Sequences as well. However, it is essential to highlight that those sequences functionally equivalent to the sequences of the above mentioned peptides are also contained in the scope of the present invention.

And, in this sense, it is important to point out that for sequences functionally equivalent it is understood those that could perform the same part, without being identical, in view of the use or application considered. Such equivalent sequences can be the result of the variability, in other words, any modification, spontaneous or induced in a sequence, whether of substitution and/or deletion and/or insertion of nucleotides/amino acids, and/or extension and/or shortening of the sequence in one of its extremities. A non-natural variability can result from techniques of genetic engineering.

It is further important to emphasize that in the present invention the use of the expression ‘at least one’ of the peptides p37 up to p94 (corresponding to SEQ ID No. 1 up to SEQ ID No. 58 respectively, or sequences functionally equivalent to the same ones) denotes said peptides individually or combinations of the same ones.

The present invention aims at the sensitive and specific detection of infections by M. leprae through a method characterized by the following stages:

(a) Collect the sample to be examined;

(b) Extract from the sample obtained in (a) the biological material of interest;

(c) Put the biological material originating from stage (b) in contact with at least one of the peptides p37 up to p94 (corresponding to SEQ ID No. 1 up to SEQ ID No. 58 respectively, or sequences functionally equivalent to the same ones):

(d) In view of the contact carried out in stage (c), by means of appropriate techniques detect antigen-specific induction of immunoinflammatory molecules and/or presence of cells responding to peptides used (or sequences functionally equivalent to the same ones).

In stage (a) previously described, the sample to be examined can be, but it is not limited to, blood. And extraction stage (b) of the biological material of interest (for instance, mononuclear cells of peripheral blood—PBMC) of the sample obtained in (a) can be carried out according to techniques known by those skilled in the art, as in the case of separation by ficoll-hypaque gradient.

In stage (c), the contact of the biological material originating from stage (b) with at least one of the peptides p37 up to p94 (corresponding to SEQ ID No. 1 up to SEQ ID No. 58 respectively, or sequences functionally equivalent to the same ones) can also be carried out according to techniques known by those skilled in the art. One example is the plating of PBMCs in plates of 96 wells of “U” bottom (BD Biosciences) and the stimulation, in vitro, with at least one of said peptides.

Stage (d) can also be carried out according to appropriate techniques, which are known by those skilled in the art, as in the case of assessment of IFN-γ production by ELISA.

This invention is also related to a kit for use in sensitive and specific detection of infections by M. leprae through the method presented in the present invention. Basically, the kit comprises in addition to at least one of the peptides p37 up to p94 (corresponding to SEQ ID No. 1 up to SEQ ID No. 58 respectively, or sequences functionally equivalent to the same ones), additives and reagents necessary for the execution of stage (c) of the present method, as in the case of: buffers, culture medium, monoclonal antibodies, enzymes and enzymatic substrata, etc., in sufficient amounts to carry out said stage (c). Optionally, it may contain protocol and manual to give instructions to the user.

In the present invention at least one of the peptides p37 up to p94 (corresponding to SEQ ID No. 1 up to SEQ ID No. 58 respectively, or sequences functionally equivalent to the same ones) can be used as active principle(s), of pharmaceutical and immunological compositions (vaccines, for instance) and for example for the treatment or prevention of infections caused by M. leprae.

The present invention is described in full detail through the examples presented below. It is necessary to emphasize that the invention is not limited to these examples, but it also includes variations and modifications within the limits in which it works.

### EXAMPLE 1

### Materials and Methods

Selection of Genes and Peptides of Mycobacterium leprae:

Comparative analyses (bioinformatics) of the genomes of Mycobacterium leprae and Mycobacterium tuberculosis, through the use of programs developed by Dr. N. G. Stoker (Department of Pathology and Infectious Diseases, Royal Veterinary College, United Kingdom) and Dr. S. T Cole (Unitè de Génétique Moléculaire Bacterienne, Instituit Pasteur, France), were applied to generate lists of genes of Mycobacterium leprae that were not present in Mycobacterium tuberculosis, or in other mycobacteria.

Foreseen proteins of Mycobacterium leprae Mycobacterium leprae (<ftp://ftp.sanger.ac.uk/pub/pathogens/leprae/>) were compared to six possible translation pictures of other mycobacterial genomes, both the ones already sequenced (Mycobacterium tuberculosis) and the ones not finalized yet (Mycobacterium avium and Mycobacterium smegmatis), by means of the use of TBLASTN of the National Center for Biotechnology Information basic local alignment search tool—BLAST, version 2.0 (Altschul S. F., T. L. Madden, A. A. Schaffer, J. Zhang, Z. Zhang, W. Miller, D. J. Lipman, 1997. Gapped BLAST and PSI-BLAST: a new generation of protein database search programs. Nucleic Acids Res. 25: 3389-3402). Preliminary data of sequences of Mycobacterium avium and Mycobacterium smegmatis was obtained online at the electronic address of The Institute for Genomic Research (on the internet at www.tigr.org).

Sequences of proteins were also used to look for coincidences in a library of non-redundant proteins of the GenBank, through the use of the protein-protein BLAST (BLASTP). The searches were performed in May 17, 2001, and all sequences with an identity index <0.01 were discarded.

Using the sequences generated by these methods, probable open target pictures of reading were selected for the production of recombinant proteins. And the probable open pictures of reading selected were evaluated as for the homology with the genomic sequences of Mycobacterium marinum (on the internet at www.sanger.ac.uk/Projects/M_marinum) or Mycobacterium smegmatis (on the internet at www.tigr.org/tdb/mdb/mdbinprotgress.html).

The sequences of peptides (15 or 19 mer) were selected as having high binding affinity to human class II HLA-DR (DRB1*0101, DRB1*0301 (DR17), DRB1*0401 (DR4w4), and DRB1*1101) or to human class I (HLA-A*0201), using the SYFPEITHI program (on the internet at www.bmi-heidelberg.com/syfpeithi).

Then, all peptides were analyzed through the BLASTP as for the homology with sequences of other proteins, using the program available for BLAST searches by almost exact short identities (on the internet at www.ncbi.nlm.nih.gov/BLAST). Peptides with significant homology degrees with genes expressed in other potential human pathogenic microorganisms were excluded, for which the sensitization of T cell could be expected (for example, Streptococcus sp., Haemophilus influenzae).

Peptides used in this study were synthesized with the amino and free carboxi-terminal portions in the scale of 1 mmol. Each one of these peptides was dissolved in distilled water, sonicated and stored at approximately −70° C., or aliquoted and relyophilized.

### Population Study for Immediate Response Experiments by Cells.

For the analysis of the response of IFN-γ to peptides of Mycobacterium leprae, a total of sixty volunteers were studied (ten in each group).

Non-treated paucibacillary patients (PB) (ten borderline tuberculoid patients—BT), non-treated multibacillary patients (MB) (three with the polar lepromatous form—LL, and seven with the borderline lepromatous form—BL) and household contacts (HC) of MB patients MB were recruited at Souza Araújo Unit (Fundacão Oswaldo Cruz—Brazil).

Non-treated PPD positive patients with lung tuberculosis (TB) were recruited at Hospital Clementino Fraga Filho/Universidade Federal do Rio de Janeiro—Brazil. Healthy individuals (who live in the city of Rio de Janeiro and with an unknown history of exposure to leprosy and tuberculosis) were included as an endemic control group (EC), while individuals living in the State of Colorado (USA) served as a non-endemic control group (NEC). The tests and procedures described in this document were approved by the Ethics Committee of Fundacão Oswaldo Cruz and by the Internal Review Board of Colorado State University.

Is it important to highlight that the secretion of IFN-γ in supernatants of cultures was used to evaluate the response concerning specific T cells, taking into consideration that it has been demonstrated that the immunity associated to mycobacterium infection requires specific activation of T cells secreting type 1 cytokines, such as IFN-γ (Gallo Men, Sampaio E., Nery Já, Morais M. O., Antunes S. L., Pessolani M. C. V., Sarno E. N. Editor: COURA, J R. Hanseníase: Aspectos Epidemiológicos, Clínicos e Imunológicos. Dinâmica das doencas infecciosas e parasitárias. 2005. Editora Guanabara Koogan S A. pages: 1383-1394; GAUDUIN M.C. Intracellular cytokine staining for the characterization and quantitation of antigen-specific T lymphocyte responses. Methods. 2006; 38(4):263-73).

Culture In Vitro of PBMC (Peripheral Blood Mononuclear Cells) Stimulated with Peptides:

The blood was collected by venous pulsation, heparinized, and PBMC isolated using Lymphoprep (Pharmacia Biotech) by density gradient, followed by centrifugation of 1000×g for 30 minutes, washing with PBS (Gibco) and resuspension in AIMV medium (Invitrogen Life Technologies) supplemented with 100 U/ml of penicillin, 100 μg/ml of streptomycin and 2 mM of L-glutamine (Sigma-Aldrich).

PBMCs (2×10⁵ cells/well) of each individual were plated, in replication, in plates of 96 wells “U” bottom (BD Biosiences) and stimulated, in vitro, with sonicated Mycobacterium leprae (20 μg/ml), individual recombinant proteins or peptides (10 μg/ml), or Enterotoxin B of Staphylococcus aureus (SEB—1 μg/mL—Sigma Aldrich). The cultures were incubated at 37° C. in 5% of CO². The supernatants were removed in the fifth day of incubation and immediately stored at −70° C. until the execution of ELISA to quantify IFN-γ.

The levels of IFN-γ were evaluated in replication, using IFN-γ DuoSet ELISA Development System Kit (R&D Systems) with a detection limit of 125 pg/ml.

Frequency Analysis of T CD4+ and CD8+ Lymphocytes Producers of IFN—in Cultures of PBMCs Stimulated with Synthetic Peptides

PBMCs (5×10⁵ cells/well) were plated in plates of 96 wells of “U” bottom in complete AIMV medium, and were stimulated with simple peptides (p51, p52, p59, p61, p65 and p69) or with a pool of these peptides, in the presence of costimulatory Abs specific for CD28 and CD49d. The cultures were maintained in an CO² incubator humidified at 37° C. for six hours, and in the last one hour 10 μg/mL of Brefeldin A was included (Sigma Aldrich). After the incubation, the cells were permeabilized, followed by fixation with 1% of paraformaldehyde in PBS. The cells were washed with buffer (0.5% BSA and 0.1% NaN³ in PBS) and marked with 2 μl of an anti-INF-γ FITC combination, anti-CD69 PE and anti-CD4 PerCP-Cy5.5 (BD Pharmingen) or 20 μl of an anti-INF-y FITC combination, anti-CD69 PE, anti CD8 PerCP-Cy5.5 and anti-CD3 APC (BD Pharmingen). The control tubes were reddened with control isotype.

### Analysis by Flow Cytometry:

The four-color cytometry results, using the FACSCalibur Flow cytometer, were obtained and analyzed by BD CellQuest Pro Software (BD Biosciences). A total of 40,000 T CD4+ cells and 10,000 T CD8+ cells were counted. A region of lymphocytes was delimited in the bi-dimensional charts of points showing levels of luminous dispersal in straight line (FSC) and in right angle (SSC) in samples of PBMCs. From this region a second region only constituted of T CD4+ cells and/or T CD3+/CD8+ cells was created. The frequencies of T CD4+ cells and/or CD3+/CD8+ cells were analyzed as for CD69 PE expression and/or IFN—in charts of points. A negative region for each population of cells was defined as isotype control. CaliBRITE “beads” for the cytometer calibration (BD Pharmingen) were used.

### Statistical Analysis:

ANOVA (Statistica, Version 6.0, Statsoft) was used to determine differences in the levels of IFN-γ induced by each peptide on PB and MB patients, HC individuals, TB patients and EC individuals. In this analysis, the cutoff was defined as twice the value of the detection limit (250 pg/ml). The analysis was followed by the Tukey test used as a Post Hoc test to compare two groups. The difference between the groups was considered significant when p was lower or equal to 0.05.

### EXAMPLE 2

### Identification of Homologies of Peptides and Proteins in Other Mycobacterium Databanks

The criterion to select many of the peptides of specific hypothetical unknown proteins, in addition to other proteins of Mycobacterium leprae was started in the beginning of 2002. And, ever since, sequences derived from projects of mycobacterium genomes have been made available, including the complete sequence of Mycobacterium bovis and the sequences almost finalized or still preliminaries of Mycobacteriuin avium, Mycobacterium marinum, Mycobacterium smegmatis, Mycobacteriun ulcerans and Mycobacterium paratuberculosis (Garnier T., K. Eighmeier, J. C. Camus, N. Medina, H. Mansoor, M. Pryor, S. Duthoy, S. Grondin, C. Lacroix, C. Monsempe, et al, 2003, The complete genome sequence of Mycobacterium bovis. Proc. Natl. Acad. Sci. USA 100: 7877-7882).

The most recent search (Aug. 25, 2005), using the BLASTP program for short segments with almost exact identities identified homologies within 58 peptides selected. Table 1 presents data related to these peptides.

TABLE 1

Specific peptides of Mycobacterium leprae having high
binding affinity for HLA molecules (Human leukocyte
antigens) MHC (Major histocompatibility complex), and
tested to evaluate IFN-γ responses.

| | | | | |
|---|---|---|---|---|
| Peptide | ID/Gene | Place | Sequence Number | Sequence |

| | | | | |
|---|---|---|---|---|
| p37 | ML0008c | aa 1-15 | SEQ ID No. 1 | MATIRTVRNLKLCNP |
| p38* | ML0008c | aa 21-35 | SEQ ID No. 2 | TRLLTVVVKQRSKAF |
| p39* | ML0008c | aa 24-38 | SEQ ID No. 3 | LTVVVKQRSKAFRPS |
| p40* | ML0008c | aa 107-121 | SEQ ID No. 4 | RVSYGSECRSGNCLR |
| p41 | ML0008c | aa 2-10 | SEQ ID No. 5 | ATIRTVRNL |
| p42* | ML0126 | aa 250-264 | SEQ ID No. 6 | LDDFLSLQRSISPNS |
| p43 | ML0126 | aa 90-104 | SEQ ID No. 7 | ATHYFEMTSDAFFAD |
| p44* | ML0126 | aa 251-265 | SEQ ID No. 8 | DDFLSLQRSISPNSY |
| p45 | ML0126 | aa 65-79 | SEQ ID No. 9 | DVKIAVDPACKLSAR |
| p46 | ML0394c | aa 117-131 | SEQ ID No. 10 | HLILRITPGIDLREL |
| p47* | ML0394c | aa 73-87 | SEQ ID No. 11 | LNQLVSLVKQIFVQQ |
| p48* | ML0394c | aa 22-36 | SEQ ID No. 12 | QLMYLIEITSETKAL |
| p49* | ML1057 | aa 10-24 | SEQ ID No. 13 | GRYYAEINSAKMYFG |
| p50 | ML1057 | aa 46-60 | SEQ ID No. 14 | VLVLVGDEAAALEQL |
| p51* | ML1057 | aa 54-68 | SEQ ID No. 15 | AAALEQLLGQTADVA |
| p52* | ML1057 | aa 59-67 | SEQ ID No. 16 | QLLGQTADV |
| p53* | ML2567 | aa 95-109 | SEQ ID No. 17 | RRTVKILRPLPSNIT |
| p54* | ML2567 | aa 72-86 | SEQ ID No. 18 | NHAVSSDFKTRSTNT |
| p55 | ML2567 | aa 128-136 | SEQ ID No. 19 | DIAARLASL |
| p56* | ML0308 | aa 17-31 | SEQ ID No. 20 | FDEYRAMFALSAMDL |
| p57 | ML0308 | aa 139-153 | SEQ ID No. 21 | THAFDLVLSSHLLFT |
| p59 | ML0398c | aa 15-29 | SEQ ID No. 23 | MLILGLLPAILPACG |
| p60 | ML0398c | aa 288-302 | SEQ ID No. 24 | AILYRKIHGQDPAER |
| p61* | ML0398c | aa 16-24 | SEQ ID No. 25 | LILGLLPAI |
| p62 | ML0678c | aa 31-45 | SEQ ID No. 26 | PYRFHSLAAIWALSP |
| p63* | ML0757c | aa 6-20 | SEQ ID No. 27 | GINLPKDELTAFGRK |
| p64 | ML0757c | aa 74-88 | SEQ ID No. 28 | VGAVRIVGGVRPQNF |
| p65* | ML1419c | aa 108-122 | SEQ ID No. 29 | EAVLLRLDGTTLEVE |
| p66* | ML1419c | aa 137-151 | SEQ ID No. 30 | QVIFRDLTTQKAAEE |
| p67* | ML1419c | aa 51-65 | SEQ ID No. 31 | SGRVTYLNPVGVKWM |
| p68* | ML1419c | aa 267-275 | SEQ ID No. 32 | LLEEGVIVL |
| p69* | ML1419c | aa 113-121 | SEQ ID No. 33 | RLDGTTLEV |
| p70* | ML1420 | aa 93-107 | SEQ ID No. 34 | MQEYRGLTSHTPCCR |
| p71* | ML1553 proS | aa 180-194 | SEQ ID No. 35 | LDIYTTLARDMAAIP |
| p72 | ML1553 proS | aa 154-168 | SEQ ID No. 36 | TIEFLWQEGHSAHIE |
| p73* | ML1829 | aa 108-122 | SEQ ID No. 37 | DAEWLKLTSLGLRPR |
| p74* | ML1915 | aa 89-104 | SEQ ID No. 38 | VKAVVDDVNAILLTGR |
| p75* | ML2177c | aa 169-183 | SEQ ID No. 39 | LQPYRLLRGGDSEYW |
| p76* | ML2498 | aa 75-89 | SEQ ID No. 40 | VGKVQGLLARLLTLP |
| p77* | ML2703 trxB | aa 331-335 | SEQ ID No. 41 | DSTDTTDWSTAMTDA |
| p78 | ML0411 | aa 264-278 | SEQ ID No. 42 | GLDSIISSASASLLT |
| p79 | ML0098 fbpC | aa 84-98 | SEQ ID No. 43 | GNAMTTLGGRGISVV |
| p80* | ML0126 | aa 125-139 | SEQ ID No. 44 | YGQVVRDVENTLRYL |
| p81* | ML0126 | aa 180-194 | SEQ ID No. 45 | GDVWKSIVHLRSTRH |
| p82* | ML0394c | aa 141-155 | SEQ ID No. 46 | KKRLTLLSGAMARRA |
| p83* | ML0394c | aa 97-111 | SEQ ID No. 47 | DATFQVIFSQHVHLS |
| p84 | ML0638 | aa 1-15 | SEQ ID No. 48 | MIDYNNVFGAGVVAA |
| p85 | ML0638 | aa 49-63 | SEQ ID No. 49 | NYEVSPIFARWPRNR |
| p86* | ML0840c | aa 287-301 | SEQ ID No. 50 | VYLYNYLLAETSHVL |
| p87 | ML0840c | aa 251-265 | SEQ ID No. 51 | YRYYRLIATTDAASP |
| p88* | ML1189c | aa 55-69 | SEQ ID No. 52 | DDIWRTLASAVITGN |
| p89 | ML1189c | aa 10-24 | SEQ ID No. 53 | FDSFDRILKARSPEA |
| p90 | ML2347 | aa 20-34 | SEQ ID No. 54 | KENVIIVAAKISWTL |
| p91* | ML2347 | aa 301-315 | SEQ ID No. 55 | LATVQYDDRRRFTKE |
| p92* | ML2452c | aa 28-42 | SEQ ID No. 56 | LQAYSNLFGRTSAMQ |
| p93 | ML2591 mce1C | aa 62-76 | SEQ ID No. 57 | GMDVGKVEALKIDGD |
| p94 | ML2596 | aa 165-179 | SEQ ID No. 58 | WASVALLTAAGVSLG |

In Table 1, the information about all genes and sequences was obtained at <on the internet at www.genolist.pasteur.fr/Leproma/>. Peptides that induced response in leprosy patients only, or contact groups (but not in TB or EC groups) appear with an asterisk. Peptides p37 up to p55, p62 up to p64, p70, p73, p74, p80 up to p83, p86 up to p89 and p92 derive from hypothetical and unknown proteins (class VI).

Peptide p55 from ML2567 (9-mer) showed 100% identity with an 8-mer from another gene of Mycobacterium leprae—ML0314 (a possible esterase), although these proteins are not related.

The peptides below (which present a high identity with peptides of Mycobacterium avium paratuberculosis K10) have the following characteristics:
p62 from ML0678c (83% of identity with one 12-mer from MAP3814c gene);
p86 and p87 from ML0840c (80% of identity with one peptide 10-mer in MAP2122 gene, and 66% of identity with one 15-mer in the same gene, respectively);
p88 and p89 from ML1189c (84% of identity with one 13-mer in MAP2344 gene, and 80% of identity with one 15-mer in the same gene, respectively).

Peptide p69 from ML1419c (9-mer) showed 100% of identity with one 8-mer from Streptomyces coelicolor. And peptides p79 from ML0098 (fbpC) and p93 from ML2591 (mcelC), which have homologous in Mycobacterium tuberculosis and were selected in order to examine the crossed responses in TB individuals, also showed high identity with sequences of peptides from Mycobacteriun avium paratuberculosis K10 (86% of identity with one 15-mer in fbcCl and 93% of identity with one 15-mer in MAP3606 gene).

A search for homologies with the four hypothetical and unknown genes of this work pointed out that although ML0008, ML1057 and ML2567 do not present ortologues in Mycobacterium tuberculosis or in other databanks of mycobacterium data available; one gene with 69% of identity with ML0126 was identified in the genome of Mycobacterium ulcerans (<http://genopole.pasteur.fr/Mulc/Burulist.html>). The six peptides of 15-mer from ML0126 tested in this work—p42 up to p45, p80 and p81—showed between 67 and 87% of identity with the sequences foreseen in Mycobacterium ulcerans. The more sequences are added to mycobacterium databanks, the more other ortologues and homologous to the hypothetical and unknown proteins of class VI of Mycobacterium leprae can be identified.

### EXAMPLE 3

### Levels of IFN-γ in Supernatants of PBMC Culture (Mononuclear Cells of Peripheral Blood) in Response to Recombinant Proteins and to Peptides

PBMC of groups of hansenian patients (PB and MB), contacts of MB (HC) patients, healthy individuals from the endemic area (EC) and healthy groups from non-endemic area (NEC) were cultivated with recombinant proteins, peptides and controls. The production of IFN-γ was measured on the fifth day of cultivation.

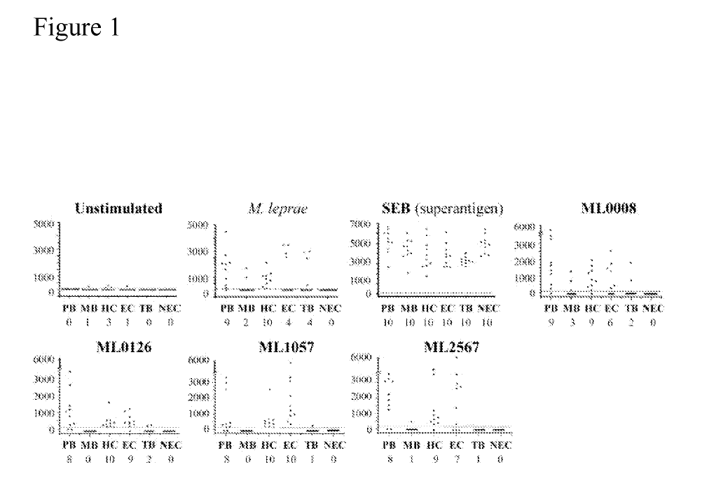

The results relative to the IFN-γ responses referring to the four recombinant proteins and to the controls and shown in FIG. 1. In addition, this FIG. 1 shows results with sonicated Mycobacterium leprae and superantigen SEB (Enterotoxine B of Staphylococcus aureus). The mean alone (non-stimulated cultures) represents the IFN-γ response in the absence of antigenic stimulus. The dotted line indicates the cutoff of 125 pg/ml of IFN-γ, which was measured in the supernatants. The total number of responses is shown below each group.

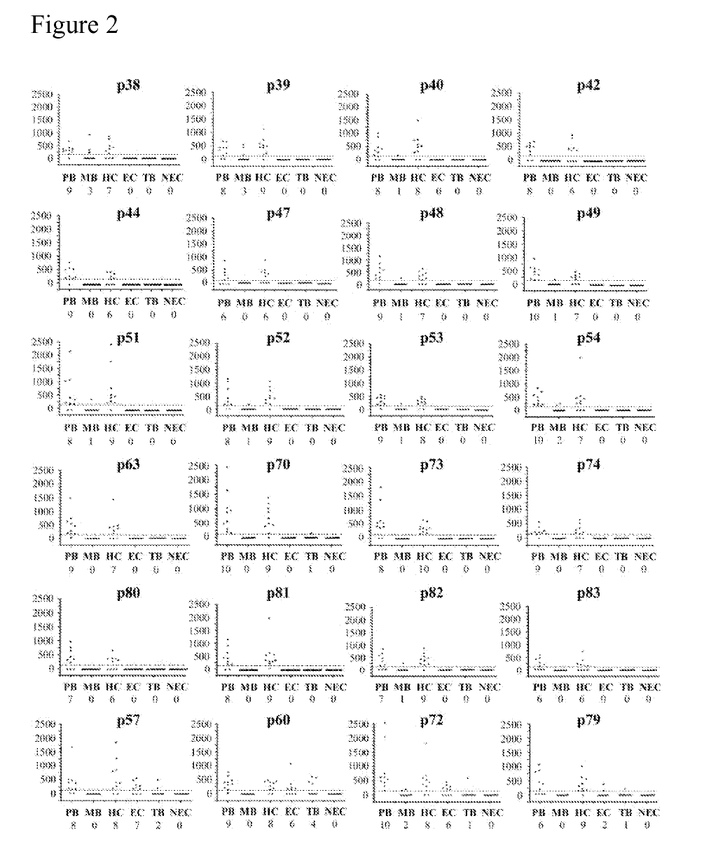

The responses that are representative of a subgroup of the 58 tested peptides in the present work are presented in FIG. 2. In this Figure, it is possible to note that 20 peptides have led to responses in groups PB and HC, but without responses in groups TB or EC. In addition, peptides p57, p60, p72 and p79 have induced responses in individuals in groups TB and EC, in addition to the responses in groups PB and HC. The dotted lines represent the cutoff of 125 pg/ml of IFN-γ, which was measured in the supernatants. The total number of responses is shown below each group.

The responses to all peptides and to the proteins from group NEC were uniformly negative, while the IFN-γ responses were lower than or, in some cases, a little above the limit of detection in all non stimulated cultures (alone mean) in all the groups. All the individuals responded well when their cells were cultivated in the presence of superantigen SEB.

The responses to four recombinant proteins were generally higher than with individual peptides; however, with responses also in groups EC and TB (ML0008, 6EC and 2 TB; ML0126, 9EC and 2TB; ML1057, 10EC and 1TB; ML2567, 7EC and no TB) (FIG. 1).

As it concerns the PBMC response of hansenian patients and contacts to most of the peptides, this has shown a large level of specificity, stimulating the production of IFN-γ in 60-100% of the 10 BT patients (PB group) and 50-100% out of the 10 individuals in the HC group. Out of 1-4 of the individuals in the MB group responded to certain peptides.

35 of the peptides did not show positive responses in groups TB or EC; however, they showed good responses in groups PB and HC (peptides indicated by an asterisk in Table 1), while 23 of the peptides presented one or more IFN-γ marked responses in groups TB or EC. Some of the peptides, which led to the most frequent responses in groups EC or TB, were those that had homologues in Mycobacterium tuberculosis, such as peptides p79 (homologous fbpD Rv3803c, 80& of identity), p93 (homologous mceIC Rv0171, 73% of identity), and p72 (homologous proS Rv2845c, 27% of identity). Other peptides that induced responses in groups EC and TB had a very low homology or not identified to their corresponding homologues in Mycobacterium tuberculosis, such as peptides p57 and p60. A few of the peptides derived from unknown hypothetical proteins, with no homologues in any database of micobacterial genes, induced unexpected responses in groups EC or TB, such as p37 of ML0008. It may be that epitopes of crossed reactivity inside these peptides are disclosed as more micobacterial sequences are added to the databases.

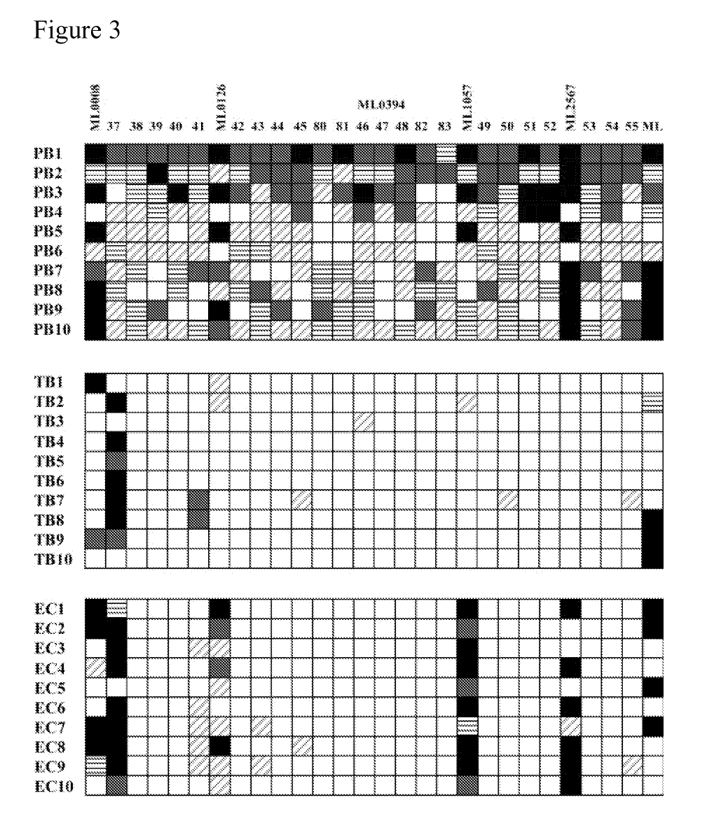

An analysis of the original data indicated that certain individuals in group PB responded well to most of the peptides, while other individuals responded weakly or did not respond in relation to many of the peptides. Those that, in groups TB and EC, did not respond to most of the peptides (FIG. 3).

In FIG. 3 the responses of groups PB, TB and EC are presented with regard to the four recombinant proteins and their respective peptides, and to peptides from ML0394, in addition to the sonicated Mycobacterium leprae. The boxes with horizontal lines indicate the relative amounts of IFN-γ detected; the blank boxes indicate an amount lower than the limit of cutoff detection (<125 pg/ml); the boxes with diagonal lines represent 125-299 pg/ml; the boxes containing dots are associated to 300-499 pg/ml; the boxes in dark grey represent 500-999 pg/ml and the black boxes indicate the results with >1000 pg/ml.

The individuals with the best responses in these groups also reacted well with the preparation of the sonicate of Mycobacterium leprae. In general, members of group HC responded similarly or better than the individuals from group PB; which was expected due to the fact that most of the HC, although having been exposed to the disease for different periods of time, are healthy and immune. So, they present responses that are a lot stronger to Mycobacterium leprae. In short, the responses in the hansenian patients, their contacts and controls, to most of the peptides, were quite specific, showing no cross reactivity, which has been reported recombinant proteins that have low homology (Geluk, A., K. E. van Meijgaarden, K. L. Franken, Y. W. Subronto, B. Wieles, S. M. Arend, E. P. Sampaio, T. de Boer, W. R. Faber, B. Naafs, and T. H. Ottenhoff. 2002. Identification and characterization of the ESAT-6 homologue of Mycobacterium leprae and T cell cross-reactivity with Mycobacterium tuberculosis. Infect. Immun. 70: 2544-2548; Geluk, A., K. E. van Meijgaarden, K. L. M. C. Franken, B. Wieles, S. M. Arend, W. R. Faber, B. Naafs, and T. H. M. Ottenhoff. 2004. Immunological cross-reactivity of the Mycobacterium leprae CFP-10 with its homologue in Mycobacterium tuberculosis. Scand. J. Immunol. 59: 66-70).

Four out of six 9-mers showed a good potential specificity. Three of these six 9-mers peptides were contained in one of the tested 15-mer peptides: p52 in p51 (ML1057); p61 in p59 (ML0398c), and p69 in p65 (ML1419c). For the pairs of peptides from ML1057 and ML1419c, both the longer 15-mer peptide and the shorter 9-mer peptide were specific, inducing good responses in groups PB and HC, and no response in groups TB, EC or NEC. For the peptides from ML0398c, shorter 9-mer peptide was more specific than longer 15-mer peptide. In a general way, 31 out of the 52 peptides presented promising specificities, while 4 out of the 6 9-mer peptides showed such a behavior. This indicates that the shorter 9-mer peptides, of which one would expect an induction of the CD8 AG-specific activation, as well as the longer peptides, which would be recognized mainly by cells T CD4+, can be employed in tests for the detection of infection by Mycobacterium leprae.

### EXAMPLE 4

### Frequency of Lymphocytes T CD4+ and CD8+ Producers of IFN-γ in Cultures of PBMCs Stimulated with Synthetic Peptides.

In order to define the phenotype of T cells responding to peptides of 15- and 9 mer, the frequencies of peptide-specific T cells CD4+ and CD8+ producers of IFN-γ, which were present in the blood of the BT patients, were analyzed by flow cytometry.

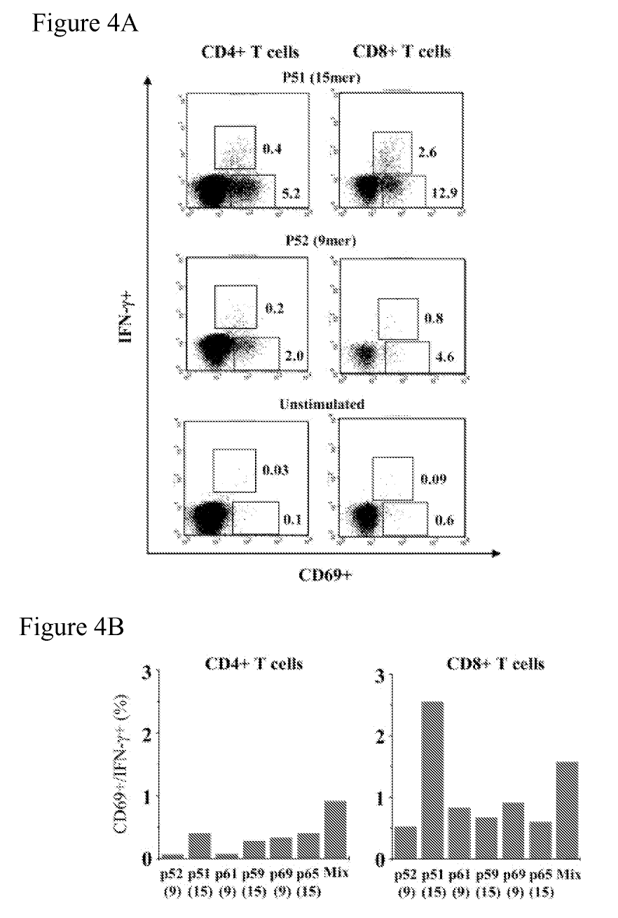

The results show high frequencies of T CD4+ e CD8+ T cells producers of IFN-γ, which were stimulated with peptides 15- or 9-mer, or the combination of same. When 9-mers and 9-mer containing 15-mers were compared, peptides 9-mer activated the CD8+ T cells in a preferential way, while peptides 15-mer induced responses in both subgroups of CD4+ e CD8+ T cells (FIG. 4).

FIG. 4 shows the frequencies of peptide-specific CD4+ and CD8+ T cells producers of IFN-γ in an BT patient ex-vivo. PBMCs were stimulated with peptides individually (p51, p52, p59, p61, p65 and p69 at 10 μg/ml each) and with a combination of same (10 μg/ml each). Six hours later, the cells were flushed by utilizing kit FastImmune CD4 (anti-IFN-γ FITC, anti-CD69 PE, and anti-CD4 PerCp-Cy5.5) or CD8 (anti-IFN-γ FITC, anti-CD69 PE, anti-CD3-APC, and anti-CD8 PerCp-Cy5.5) of BD Biosciences. The analysis was performed in the lymphocytes after the CD4+ T cells were selected (40,000 events) and/or CD8+ T cells (10,000 events) in a FACSCalibur flow cytometer. FIG. 4A exhibits a graph that shows the frequency of CD4+/CD69+ and/or CD8+/CD69+ T cells producers of IFN-γ relative to the responses induced by peptides p51 and p52. As it concerns FIG. 4B, this presents the bars referring to the frequencies of the CD4+/CD69+ and/or CD8+/CD69+ T cells producers of IFN-γ corresponding to the responses brought about by peptides 9-mer p52, p61 and p69, and those relative to peptides 15-mer p51, p59 and p65 containing peptides 9-mer, respectively, in addition to a combination of these six peptides.

### EXAMPLE 5

### Statistical Analysis of the Responses of the Groups to Individual Peptides.

The levels of IFN-γ, induced by individual peptides in the different groups, were compared statistically in accordance with the employment of the ANOVA test. Tukey's test was used as a post hoc test, in order to evaluate significant differences between two groups.

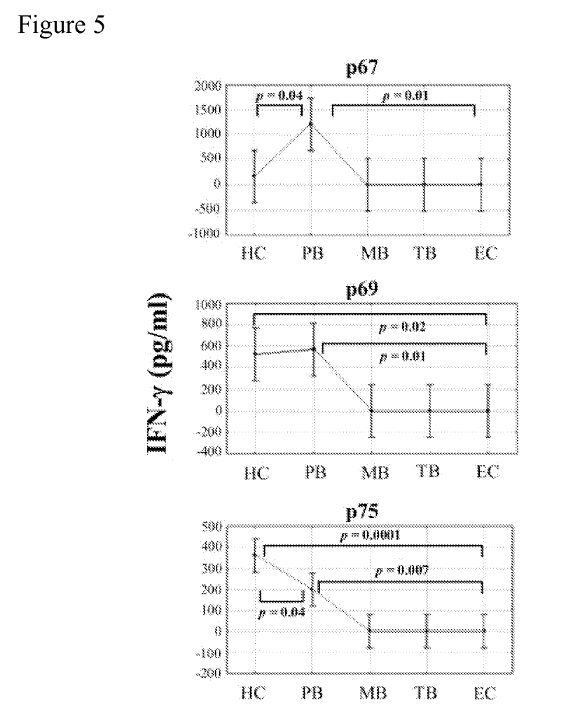

When individually analyzed, three types of responses could be distinguished, which are represented in FIG. 5 by peptides p67, p69 and p75.

FIG. 5 is relative to the ANOVA of the levels of IFN-γ induced by synthetic peptides of Mycobacterium leprae. The vertical bars represent intervals of 0.95. The values ofp are the following: p67, p=0.00003; p69, p=0.00003; and p75, p=0.00008. Tukey's test was used as a post hoc test to evaluate the significant different between the groups studied, and values of p≦0.05 are indicated in the graphs; PB, Paucibacilar Patients; MB, Multibacilar Patients; TB, Patients with tuberculosis; and EC, EC healthy.

The graphs present average values for each group, and the intervals of 0.95 were adjusted through ANOVA. The IFN-γ responses to p69 were significantly higher in HC (p=0.02) and in PB (p=0.01), as opposed with those seen in groups MB and in the control groups (TB and EC). 29 out of the 35 peptides, which did not show positive responses in groups TB and EC, induced this kind of response and may be employed to detect both HC and PB. Peptide p75, on the other hand, induced levels of IFN-γ which were significantly higher in HC, when compared with PB p=0.04) and MB (p=0.001) and the control groups (p=0.001). Three other peptides (p68, p91 and p92) induced the same kind of response and may be employed to distinguish infected and healthy individuals. Finally, responses IFN-γ to p67 were higher in PB (p=0.01), when compared with HC (p=0.04) and those seen in MB (p=0.01) and in the control groups (p=0.01), and may be employed in the detection of individuals with the paucibacilar form of Hansenosis.

Any way, according to recommendation by the Programas Nacionais de Controle [National Hansenosis Programs], two tools need to be urgently developed so as to allow the adequate control of this endemy: 1) tests which enable the specific detection of this infection, still in its early stages, thus allowing for the interruption in the transmission and the emergence of physical disability; and 2) a more effective vaccine than BCG, which is the only vaccine currently available against this micobacteriosis. Since the infections by micobacteria induce in the host an immune response of the cellular type, a cutaneous test, which classically measures this type of response, could be the type of immunological test selected for the evaluation of previous exposure and/or active infection by these pathogens.

Indeed, the tuberculinic test or the PPD test (Proteina purificada derivada de Micobacterium tuberculosis) [Purified Protein Derived from Micobacterium tuberculosis] has been used for more than 50 years, supporting the diagnosis of tuberculosis. PPD, however, consists of a crude extract of bacterial proteins and, thus, is of low specificity. On the other hand, Hansenosis lacks a test similar to the PPD, which measures the previous exposure/active infection. The cutaneous test for lepromin, used up to this day, is based on a purified antigenic preparation and does not constitute a classic reaction of DTH (late hypersensitivity), being read after 21-28 days—test of Mitsuda (Mitsuda, K. 1919. On the value of a skin reaction to a suspension of leprosy nodules. Jap. J. Der. Urol. 19: 697-708; Fernandez, J. M. M. 1940. The early reaction induced by lepromin. Int. J. Lepr. 8: 1-14). The same evaluates the need to develop a immune cellular response against M. leprae, but not the previous exposure to the bacteria.

Therefore, in the present work, a panel of recombinant antigens and synthetic peptides of M. leprae has been used. These were identified as of compared genomics, with bioinformatics tools, in search of appropriate reagents to make up an immunological test specific for the detection of the infection by M. leprae. For this study, individuals with different levels of exposure to M. leprae were included in order to assess both the level of antigenicity and the specificity of the new reagents. In addition to the Hansenosis patients with the several forms of MB and PB, as well as family contacts of MB patients, groups that exhibit high exposure to the bacillus, individuals with pulmonary tuberculosis, healthy individuals from the endemic area with previous exposure unknown to M. leprae and healthy individuals from non endemic area were studied for the evaluation of the level of crossed reactivity of the new antigens.

Four recombinant proteins and 58 synthetic peptides of decapentamers and nanomers were tested and a surprising datum was their high level of antigenicity, all being identified by at least a few of the individuals infected by M. leprae, suggesting that the criteria used for the selection of antigenic molecules were appropriate. In fact, studies have shown that bioinformatics can increase the efficiency when selecting epitopes in micobacterial antigens (Vordermeier H. M., Whelan A., Cockle P. J., Farrant L., Palmer N., Hewinson R. G. Use of synthetic peptides derived from the antigens ESAT-6 and CFP-10 for differential diagnosis of bovine tuberculosis in cattle. Clin Diagn Lab Immunol. 2003; 8(3):571-8). Several programs (such as TEPITOPE, Algorhythm and ProPred) have been used to identify peptides capable of binding to MHC molecules. Programs TEPITOPE and ProPred were used to identify ligands of HLA-DR derived from tumor and endogenous proteins involved in auto-immune diseases (Manici S., Sturniolo T., Imro M. A., Hammer J., Sinigaglia F., Noppen C., Spagnoli G., Mazzi B., Bellone M., Dellabona P., Protti M.P. Melanoma cells present a MAGE-3 epitope to CD4(+) cytotoxic T cells in association with histocompatibility leukocyte antigen DR11. J. Exp. Med. 1999; 189(5):871-6).

The responses to most of the peptides (35 out of 58, or 60%) were considerably specific, leading to positive responses only in groups PB and HC, and not in the control groups (or in rare cases, above the limit of detection of 125 125 pg/ml of IFN-γ). It is also relevant to emphasize the capacity of a number of peptides (p67, p68, p75, p91 and p92) to discriminate individuals with known infection by Mycobacterium leprae (patients PB) from those individuals exposed to said microorganism, but healthy (those who have not developed a symptom of the disease yet—group HC). Therefore, the results presented herein show the potential of the synthetic peptides for the detection of infection by M. Leprae in latent forms or early phases of the active disease.

According to the present invention the sequences are listed below.

We claim:

1. Selection method of peptides from proteins or regions of proteins belonging to the genome of Mycobacterium leprae characterized by being based on the application of algorithms for the detection of regions that combine with human molecules of histocompatibility and which present the capacity to induce an immune response in individuals exposed to Mycobacterium leprae.

2. Peptide characterized by containing one of the sequences as described in SEQ ID No. 1 up to SEQ ID No. 58, or sequences functionally equivalent to the same ones.

3. Peptide characterized by presenting one of the sequences as described in SEQ ID No. 14, 15, 22, 24, 28, and 32, or sequences functionally equivalent to the same ones, according to claim 2.

4. Detection method of infections by Mycobacterium leprae characterized by the following stages:
(a) collect the sample to be examined;
(b) Extract from the sample obtained in (a) the biological material of interest;
(c) Put the biological material originating from stage (b) in contact with at least one of the peptides p37 up to p94 (corresponding to SEQ ID No. 1 up to SEQ ID No. 58 respectively, or sequences functionally equivalent to the same ones);
(d) In view of the contact carried out in stage (c), by means of appropriate techniques detect antigen-specific induction of immunoinflammatory molecules and/or presence of cells responding to peptides used (or sequences functionally equivalent to the same ones).

5. Method characterized by the fact that the sample to be examined in stage (a) is blood, according to claim 4.

6. Method characterized by the fact that the biological material of stage (b) is mononuclear cells of peripheral blood/PBMC or supernatants of their cultures, according to claim 4.

7. Method characterized by the fact that stage (c) is carried out by means of plating of PBMCs into appropriate disposable flasks and of stimulation, in vitro, with at least one of the peptides described in claim 2, according to claim 4.

8. Method characterized by the fact that stage (d) is carried out by means of assessment of IFN-γ production by ELISA or similar methods, according to claim 4.

9. Kit for use in the detection of infections by Mycobacterium leprae according to the method described in claim 4, characterized by the fact that it comprises at least one of the peptides p37 up to p94 (corresponding to SEQ ID No. 1 up to SEQ ID No. 58 respectively, or sequences functionally equivalent to the same ones), additives and reagents necessary for the execution of stage (c) of the present method, and optionally protocol and manual to give instructions to the user.

10. Kit as described in claim 9 characterized by the fact that it comprises at least one of the peptides p51, p52, p59, p61, p65 e p69 (described by means of SEQ ID No. 14, 15, 22, 24, 28 and 32 respectively, or sequences functionally equivalent to the same ones), additives and reagents necessary for the execution of stage (c) of the present method, and optionally protocol and manual to give instructions to the user.

11. Kit as described in claim 9 characterized by the fact that additives and reagents are buffers, culture medium, monoclonal antibodies, enzymes and enzymatic substrata, for instance.

12. Pharmaceutical or immunological compositions characterized by containing as active principle(s) at least one of the peptides p37 up to p94 (corresponding to SEQ ID No. 1 up to SEQ ID No. 58 respectively), or sequences functionally equivalent to the same ones.
